(19) United States
(12) Patent Application Publication
(10) Pub. No.: US 2015/0265792 A1
(43) Pub. Date: Sep. 24, 2015

(54) VENTILATING BITE BLOCK FOR USE IN ENDOSCOPY PROCEDURES

(71) Applicant: THE TRUSTEES OF THE UNIVERSITY OF PENNSYLVANIA, Philadelphia, PA (US)

(72) Inventors: Basavana Gouda Bharamana Goudra, Cherry Hill, NJ (US); Divakara Gouda, Cherry Hill, NJ (US)

(21) Appl. No.: 14/441,201

(22) PCT Filed: Oct. 23, 2013
(86) PCT No.: PCT/US13/66374

§ 371 Date: May 7, 2015

Related U.S. Application Data
(60) Provisional application No. 61/726,187, filed on Nov. 14, 2012.

(51) Int. Cl. A61M 16/04; A61B 1/015; A61B 17/34; A61B 1/00; A61M 16/08; A61B 17/02

(52) U.S. Cl. CPC A61M 16/0493; A61M 16/0833; A61M 16/0434; A61B 17/0218; A61B 17/3423; A61B 1/00154; A61B 1/00082; A61B 1/00091; A61B 1/015; A61B 2017/345; A61B 2017/3429

(57) ABSTRACT

A bite block for facilitating upper GI endoscopy procedures includes a body member that defines a first passageway, channel or lumen and a second passageway, channel or lumen. The first passageway is provided with a diaphragm for forming an airtight seal with an endoscope. The second passageway is connectable to a breathing apparatus for ventilating a patient. The body member includes a flange with a seal about a periphery thereof for engaging the face of a patient in an airtight fit about the mouth of the patient. Such a device facilitates the performance of upper endoscopic procedures by maintaining the integrity of the airway. The airway is spaced from the endoscope deployment pathway and can be maintained despite the movement of the endoscope.

### BACKGROUND OF THE INVENTION

This invention relates to a bite block that provides an airway, for particular use in endoscopic procedures. This invention also relates to an associated endoscopic medical method.

The number of upper gastro-intestinal (GI) endoscopy procedures done under anesthesia has increased remarkably in recent years. The increase is particularly noticeable in developed countries and especially in the United States. Availability of propofol, an intravenous anesthetic agent, and ultra-short acting opioids such as remifentanil are responsible for this increase in anesthesia during upper GI endoscopy procedures. The complexity of the procedures, both therapeutic and diagnostic, makes anesthesia inevitable for many procedures. Changing population demographics is another contributing factor. Many endoscopic therapeutic interventions that were considered experimental only a few years ago are used routinely especially in the treatment of some GI cancers.

Again aging population also increases patients' co-morbidity, making it very risky for the endoscopist to administer conscious sedation.

Administration of anesthesia for these procedures is challenging. Typically this involves a careful preoperative anesthetic evaluation especially with respect to potential airway difficulties. In the procedure room the patient is attached to monitors including an oxygen saturation monitor. An opioid, usually fentanyl, is administered followed by propofol. The administration of correct doses of propofol, that render the patient unconscious, yet retain the ability to self-ventilate and allow the endoscopist to introduce the upper GI endoscope without the patient coughing, is the most challenging aspect of this kind of anesthesia. It is further complicated by the three-fold pharmacokinetic and pharmacodynamic variation. Frequently, patients are either over-sedated (hence apnec with danger of severe desaturation) or under sedated (with coughing and ineffective ventilation again risking severe desaturation). Frequently, this requires withdrawal of the endoscope and institution of rescue medications (usually more propofol) or alternate ventilation strategies. Although they are effective in majority of the cases, occasional major morbidity and mortality can occur. This is especially true in ERCP's (endoscopic retrograde cholangiopancreatography) as they are frequently done in prone position.

A recent ASA (American Society of Anesthesiologists) closed claim analysis showed that about half of all anesthesia deaths occurring outside of the operating room are in GI endoscopy and half of these GI endoscopy anesthesia related deaths are airway related. Indeed many anesthesiologists are reluctant to work in this area because of the likelihood of an insecure airway. Anesthesiologists always like to be in complete control of the airway and in upper GI endoscopy procedures the anesthesiologists are needed to share the airway with an endoscopist.

In the management of potentially fatal airway problems, anesthesiologists occasionally use existing devices to make the procedures safe from both anesthetic and endoscopy standpoints. Generally, it is important to preoxygenate these patients to gain more time to address any apnea or coughing issues that lead to rapid desaturation.

A few particular methods for managing potential airway problems include (1) routine use of a nasopharyngeal airway, (2) use of high-frequency jet ventilation as a rescue method of positive pressure ventilation via a nasopharyngeal airway, and (3) use of a nasopharyngeal airway inserted in the mouth and connected to the breathing system.

A nasopharyngeal airway is utilizable in the absence of any contraindications and where atraumatic insertion is possible. This airway is in turn attached to a Mapelson breathing system and a 100% oxygen source. As a result, oxygen saturation is maintained in spite of hypoventilation. This also allows some degree of positive pressure ventilation, however unreliable and ineffective especially when needed due to leakage in the mouth, the same nostril and/or the other nostril.

High-frequency jet ventilation has the drawbacks of being cumbersome, not tested and frequently unavailable. Also most anesthesiologists are not trained in its use. Inserting a nasopharyngeal airway in the mouth provides a high oxygen source but does not allow positive pressure ventilation owing to leaks.

### SUMMARY OF THE INVENTION

The present invention aims to provide a device for improving patient ventilation during upper GI endoscopic procedures. Utilizing this device, an anesthetic practitioner can reduce the afore-described problems.

The invention provides a ventilating bite block that maintains the patient's airway while enabling endoscope insertion. A bite block in accordance with the present invention includes a body member that defines a first passageway, channel or lumen and a second passageway, channel or lumen. The first passageway is provided with at least one first sealing element such as a diaphragm for forming an airtight seal with an endoscope. The second passageway is connectable to a breathing apparatus for ventilating a patient. The body member of the bite block includes a flange with a seal for engaging the face of a patient in an airtight fit about the mouth of the patient.

Such a device facilitates the performance of upper GI endoscopic procedures by maintaining the integrity of the airway. The airway is spaced from the endoscope deployment pathway and can be maintained despite the movement of the endoscope.

A bite block in accordance with the present invention more particularly comprises a body member that includes a first tubular element defining a first passageway between a proximal side and a distal side of the body member. The first tubular element is insertable into a patient's mouth between the teeth of the upper jaw and lower jaw. The body member further includes a flange portion connected to a proximal end of the first tubular element. The flange portion is disposable outside and adjacent to the patient's mouth. A second tubular element is connected to the flange portion and defines a second passageway between the proximal side and the distal side of the body member. The second tubular element is connectable to a breathing apparatus for directing or guiding oxygen to the patient. At least tie or coupling member such as an elongate flexible strap or band (or a bracket or helmet) is provided and is connectable at least indirectly to the body member to attach the body member to the patient. At least one sealing element is provided on a distal (or patient-facing) surface of the flange portion for engaging the patient's face about the mouth in an at least substantially airtight fit. A diaphragm is attached to the first tubular element and is disposed across the first passageway for forming an airtight seal with an endoscope insertable through the first passageway.

A bite block in accordance with the present invention is a ventilating bite block. It provides an oral airway that enables positive pressure ventilation and the maintenance of oxygen saturation in spite of hypoventilation. The diaphragm permits easy scope introduction and prevents any significant leaks even under positive pressure ventilation.

A bite block in accordance with the present invention may further comprise a third tubular element connected at least indirectly to the flange portion and defining a third passageway between the proximal side and the distal side of the body member. The second tubular element and the third tubular element may be removably fitted to the bite block body member and are optionally parts of a Y-port. The second tubular element and the third tubular element, whether separate or parts of the same Y-port, preferably intersect and communicate with the first tubular element.

Pursuant to another feature of the present invention, the bite block further comprises an airway extension in the form of a substantially rigid but partially flexible arcuate slat projecting from the distal side of the body member. The airway extension is optionally formed with a longitudinally extending lumen, a coupling port at one end for connecting the lumen to a source of fluid, and a nozzle or spray outlet at an opposite end. The airway extension may be permanently connected to the body member of the bite block or may be removably affixed thereto. In the latter case the body member is provided with a connection component (a self-sealing slit, hooks, brackets, screws, etc.) for enabling a detachable coupling of the airway extension to the body member. The airway extension can be of various shapes (tubular, semitubular or can have an additional semicircular lumen projecting from the middle of the surface and extending all the way to the tip) These modifications can help in positioning of a fiberoptic bronchoscope.

A bite block in accordance with the present invention may alternatively comprise a tubular airway element, i.e., an oropharyngeal or internal ventilation tube, exemplarily made of a partially flexible soft polymeric material, which is connectable to the second tubular element to further define the second passageway as extending into the patient's pharynx to the entrance to the patient's trachea. This tubular member may be provided with a preformed angle or arc for oropharyngeal contour matching.

Preferably the tubular airway element is rigidly connected to the flange portion of the body member. The rigid connection is a feature of manufacture and may be implemented by forming the second tubular element as an integral and unitary part of the body member. For instance, the body member of the bite block may be formed by a single molding process to include the first and the second tubular elements as integral parts of a unitary structure of a homogeneous polymeric material. Alternatively, the airway tubular element may be separately formed and fixed to the flange, for instance, by adhesive, heat welding or ultrasonic welding.

The tubular airway element may extend at least partially in a proximal direction away from the flange. This is in contrast to the tubular element of the endoscope deployment passageway, which extends in a distal direction from the flange. As used herein, the terms “proximal” and “distal” are to be understood in relation to the endoscopist rather than the patient. Accordingly, the word “proximal” denotes those facets of the bite block proximate to the endoscopist, while the word “distal” denotes those facets of the bite block that extend away from the endoscopist.

The first tubular element, defining the endoscope deployment pathway, is preferably made of an at least semi-rigid material, while the flange portion may be made of a partially flexible material.

The first passageway is preferably but not necessarily circular in cross-section, as endoscopes are typically cylindrical.

Pursuant to another feature of the present invention, the bite block additionally comprises an outer base member, the body member being removably attachable to the base member, the tie or coupling member being connected to the base member.

A medical method in accordance with the present invention utilizes a bite block with a body member defining a first passageway and a second passageway, the first passageway being provided with at least one first sealing element, the body member including a flange with at least one second sealing element. The method includes attaching the body member to a patient so that the flange is disposed outside and adjacent to the patient's mouth and so that the second sealing element engages the patient's face about the patient's mouth to form an airtight seal between the body member and the patient's face. The method additionally includes connecting the second passageway to a breathing apparatus and thereafter operating the breathing apparatus to ventilate the patient. An endoscope is inserted through the first passageway, with the first sealing element forming an airtight seal about the outer surface of the endoscope shaft.

Optionally, the method may further include attaching an oropharyngeal or internal ventilation tube to the body member so that the tube communicates with the second passageway. This internal ventilation tube or tubular airway extension member is inserted into the patient's pharynx to the entrance to the patient's trachea.

The method typically includes operating the endoscope to view internal tissue structures of the patient while operating the breathing apparatus to ventilate the patient and maintaining the body member in airtight engagement with the patient's face and the endoscope.

The internal ventilation tube lies in the oropharynx and away from the path of the endoscope and is also distant from the larynx for avoiding laryngeal stimulation and precipitation of coughing and laryngospasm.

The second tubular element—for the ventilation pathway—is typically 15 mm in size for receiving standard anesthesia connectors, for instance, of the elbow variety, which in turn link to standard 22 mm adapters.

The bite block of the present invention potentially makes the administration of anesthesia for upper endoscopy procedures safe and effective. Patients can be sedated more deeply than is done currently, without fear of losing the airway. Of course, extreme vigilance should be maintained in the administration of any anesthesia.

### BRIEF DESCRIPTION OF THE DRAWINGS

FIG. 1 is a schematic front or proximal-side elevational view of a ventilating bite block for upper GI endoscopic procedures, in accordance with the present invention.

FIG. 2 is a schematic bottom plan view of the bite block of FIG. 1, showing in phantom lines an endoscope shaft and an elbow anesthetic connector and also showing an optional oropharyngeal or internal ventilation tube utilizable with the bite block.

FIG. 3 is a schematic perspective view, on a smaller scale, of the bite block of FIGS. 1 and 2.

FIG. 4 is a schematic side perspective view of an alternative oropharyngeal or internal ventilation tube utilizable with the bite block of FIGS. 1-3.

FIG. 5 is a schematic side elevational view, partially in cross-section, of the bite block of FIGS. 1-3 as used with the oropharyngeal or internal ventilation tube of FIG. 4.

FIG. 6 is basically a schematic front or proximal-side elevational view of another ventilating bite block for upper GI endoscopic procedures, in accordance with the present invention, utilizable with an endoscope shaft as shown in FIG. 1 and with an oropharyngeal or internal ventilation tube as shown in FIG. 5 and having a bellows-type anesthetic connector.

FIG. 7 is a schematic top plan view of the ventilating bite block of FIG. 6.

FIG. 8 is a schematic rear or distal-side elevational view of the ventilating bite block of FIGS. 6 and 7.

FIG. 9 is a schematic top and rear side perspective view of a further ventilating bite block for upper GI endoscopic procedures, in accordance with the present invention, showing an airway-maintenance extension.

FIG. 10 is a schematic rear elevational view, partially in perspective, of the ventilating bite block of FIG. 9.

FIG. 11 is a schematic side elevational view of an airway extension member in accordance with the present invention utilizable in the ventilating bite block of FIG. 1-5, 6-8 or 9-10.

FIG. 12 is a schematic front and tope side perspective view of a ventilating bite block assembly in accordance with the present invention.

FIG. 13 is a schematic front and top perspective view, on a substantially reduced scale, of yet another ventilating bite block in accordance with the present invention.

### DETAILED DESCRIPTION

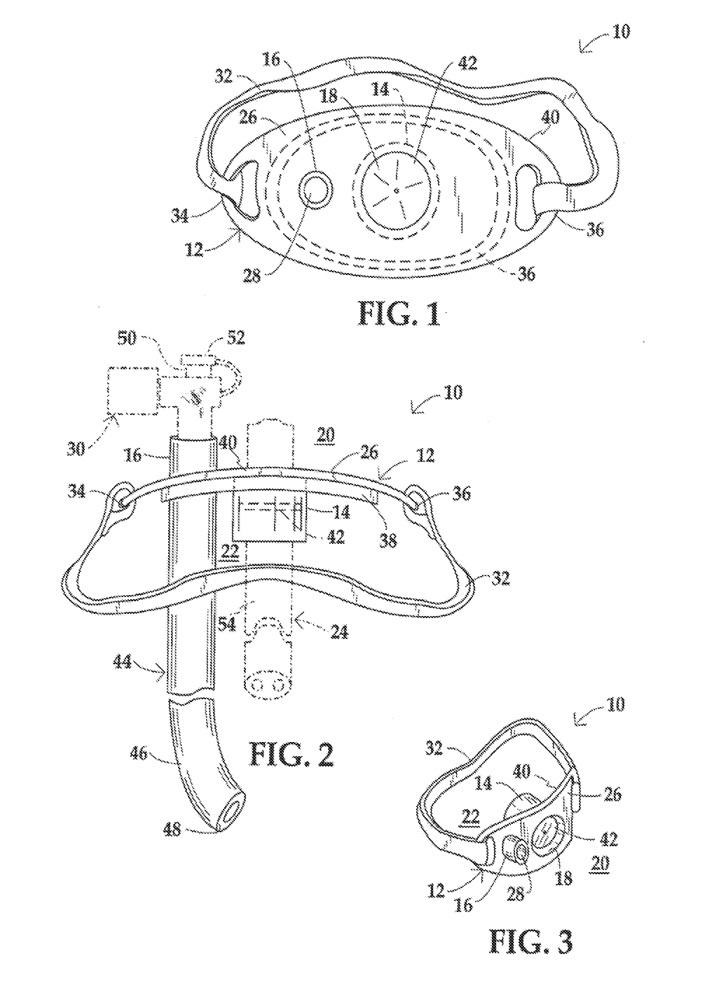

As illustrated in FIGS. 1-5, a ventilating bite block 10 for facilitating upper gastro-intestinal (GI) endoscopic procedures comprises a body member 12 that includes a first tubular element 14 and a second tubular element 16. Tubular element 14 defines a first passageway, channel, or lumen 18 between a proximal side 20 and a distal side 22 of body member 12. Tubular elements 14 and 16 are insertable into a patient's mouth between the teeth of the upper jaw and lower jaw. Passageway 18 serves as a deployment pathway for an endoscope insertion shaft 24.

Body member 12 further includes an arcuate flange 26 connected to a proximal end of tubular element 14. Flange 26 is disposable outside and adjacent to the patient's mouth. The second tubular element 16 is also connected to flange 26 and defines a second passageway, channel or lumen 28 that serves to maintain an airway for ventilating the patient. Tubular airway element 16 is connectable to a breathing apparatus (not shown), for example, via an elbow joint or channel member 30 for directing or guiding oxygen to the patient.

Bite block 10 includes at least one elongate flexible tie member 32 such as a strap or elastic band that is connectable to body member 12 at left and right ends 34 and 36 thereof. Strap 32 is extendible about the patient's head or neck for attaching body member 12 to the patient.

At least one sealing element 38 is fastened to a distal (or patient-facing) surface of flange 26 for engaging the patient's face about the mouth in an at least substantially airtight fit. Sealing element 38 extends along the entire periphery or outer edge 40 of flange 26. Sealing element 38 may take any form that provides an effective seal with human skin. Accordingly, sealing element 38 may be an oval ring or gasket made of closed cell foam or polymeric material. Alternatively, the sealing element may take the form of an air-filled oval tube of a flexible and gas-impermeable polymeric composition. In another implementation, the sealing element is a gel-filled oval ring contained by a liquid-permeable bladder. In all of these embodiments, sealing element 38 may be fastened to flange 26 via adhesive.

Within endoscope passageway 18, tubular element 14 is provided with one or more sealing elements each in the form of a diaphragm 42. Diaphragm 42 is attached to tubular element 14 and is disposed across passageway 18 for forming an airtight seal with endoscope insertion shaft 24. Other types of seals such as annular air-, water-, or gel-filled bladders or inflatable cuffs may replace or supplement diaphragm 42.

Passageway 28 of tubular element 16 partially defines an oropharyngeal airway that, together with diaphragm 42 and sealing element 38, enables positive pressure ventilation and the maintenance of oxygen saturation in spite of hypoventilation. Diaphragm 42 permits easy scope introduction and prevents any significant leaks even under positive pressure ventilation.

As shown in FIG. 2, bite block 10 may optionally include an oropharyngeal or internal ventilation tube 44 made of a partially flexible soft polymeric material and provided with a bend 46 for facilitating deployment. Ventilation tube 44 is connectable to tubular airway element 16 to extend airway passageway 28 into the patient's pharynx to the entrance to the patient's trachea. Bend 46 is a preformed angle or arc that facilitates oropharyngeal placement so that upon proper deployment a distal tip 48 of tube 44 is located at an upper end of the patient's trachea.

Preferably tubular airway element 16 is rigidly connected to flange 26. This rigid connection is implemented during manufacture and may be accomplished by molding tubular element 16 integrally and unitarily with body member 12.

Tubular airway element 16 extends at least partially in a proximal direction away from flange 26, as shown in FIGS. 2 and 3. Tubular element 14 preferably, but not necessarily, extends solely in a distal direction from flange 26. The word “proximal” is intended to denote those facets of bite block 10 that face the endoscopist, while the word “distal” denote those facets of the bite block that face the patient.

Tubular airway element 16 preferably takes the form of a 15 mm connector for receiving elbow anesthesia connector 30 exemplarily in a press-lock or friction fit. Connector 30 may be provided with a port 50 for coupling with a tidal sample tube (not shown). Port 50 has a removable cap 52 for closure when not in use.

Tubular element 14, defining the endoscope deployment pathway, is preferably made of an at least semi-rigid material, while flange 26 may be made of a semi-rigid, rigid or partially flexible material. Passageway 18 is preferably but not necessarily circular in cross-section, as endoscopes are typically cylindrical.

In a endoscopic diagnostic or therapeutic method utilizing bite block 10, body member 12 is attached to a patient so that (a) flange 26 is disposed outside and adjacent to the patient's mouth, (b) tubular element 14 extends into the patient's mouth between the upper and lower jaws, and (c) sealing element 38 engages the patient's face about the mouth to form an airtight seal between body member 12 and the patient's face. Tubular element 16 is operatively connected to a breathing apparatus (not shown) via elbow joint or channel member 30 so that passageway 28 communicates on one side with the breathing apparatus and on an opposite side with the trachea and lungs of the patient. The breathing apparatus is then operated to ventilate the patient via bite block 10. Endoscope shaft 24 is inserted through passageway 18, with diaphragm 42 forming an airtight seal about an outer surface 54 of endoscope shaft 24. Optionally, the method may further include attaching oropharyngeal or internal ventilation tube 44 to body member 12 so that the tube communicates with and extends airway passageway 28. Internal ventilation tube or tubular airway extension member 44 (similar to a nasal trumpet) is inserted into the patient's pharynx to the entrance to the patient's trachea and may be inserted part way to that location prior to attachment to body member 12, for instance, in a force-lock or friction fit. Ventilation tube 44, if used, lies in the oropharynx and away from the path of the endoscope 24 and is also distant from the larynx for avoiding laryngeal stimulation and precipitation of coughing and laryngospasm.

The method typically includes operating the endoscope to view internal tissue structures of the patient while operating the breathing apparatus to ventilate the patient and maintaining the body member in airtight engagement with the patient's face and the endoscope.

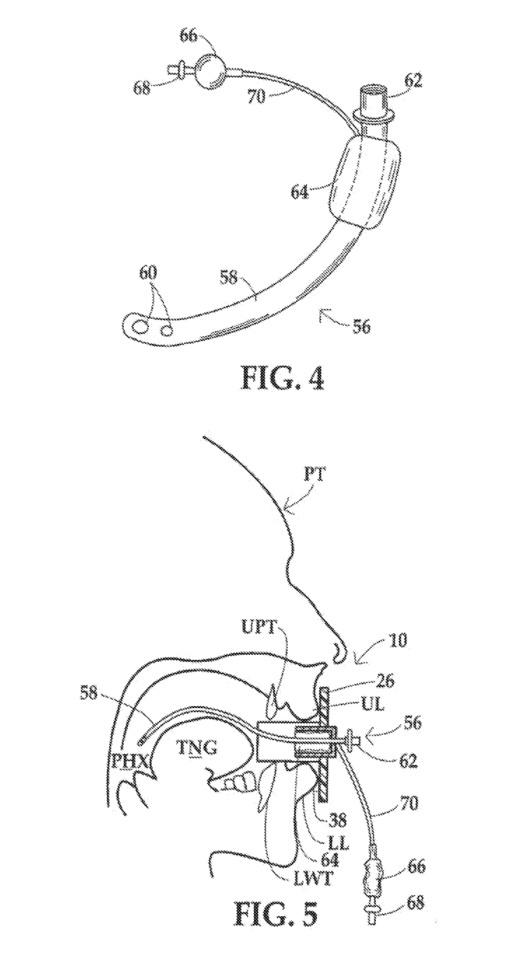

As depicted in FIGS. 4 and 5, bite block 10 may be used with an alternative oropharyngeal or internal ventilation tube assembly 56 comprising a ventilation tube member 58 provided at a distal end with a plurality of oxygen-egress apertures 60 and at a proximal end with a 15 mm connector 62 and an inflatable cuff 64. Cuff 64 is fixed along an inner side to ventilation tube member 58 and is coupled to a standard manually squeezable inflation bulb 66 via a catheter 70. Connector 60 is configured for receiving elbow anesthesia connector 30 (FIG. 2) exemplarily in a press-lock or friction fit. Connector 60 may be integrally molded and unitary with ventilation tube member 58 or permanently connected thereto exemplarily via thermal or ultrasonic welding.

As discussed hereinabove, tubular endoscope member 14 is inserted between upper teeth UPT and lower teeth LWT of a patient PT while flange 26 is placed adjacent to the patient's lips UL and LL with sealing element 38 in airtight engagement with lips UL and LL.

In one method of using oropharyngeal or internal ventilation tube assembly 56 with bite block 10, ventilation tube member 58 may be inserted through tubular airway member 16 after the installation of the bite block in the mouth of the patient. Ventilation tube member 58 is inserted into the patient's pharynx PHX over the tongue TNG to the entrance to the patient's trachea. At the onset of deployment of ventilation tube assembly 56, cuff 64 is in a deflated or collapsed pre-deployment configuration (not separately shown). Upon proper positioning of ventilation tube member 58, cuff 64 is located at least partially within tubular airway member 16. Bulb 66 is then squeezed to inflate cuff 64 to secure ventilation tube assembly 56 to bite block 10 and to form an airtight seal between ventilation tube member 58 and tubular airway member 16.

In an alternative approach, ventilation tube assembly 56 is first coupled to bite block 10, via inflation of cuff 64, and then the entire assembly is placed so that ventilation tube member 58 extends to the patient's trachea and bite block is seated as described above in the mouth of the patient PT. In either case, adjustments in the position of ventilation tube member 58 may be accomplished by partially deflating cuff 64, sliding ventilation tube member 58 relative to the bite block 10, and re-inflating cuff 64 thereafter. Bulb 66 may be provided with a manually actuatable valve 68 for deflation of cuff 64.

It is to be observed that ventilation tube assembly 56 may be used separately from independently from bite block 10. For instance, ventilation tube member 58 may be inserted through a nasal passage to establish and maintain a nasal airway, optionally with the use of bite block 10.

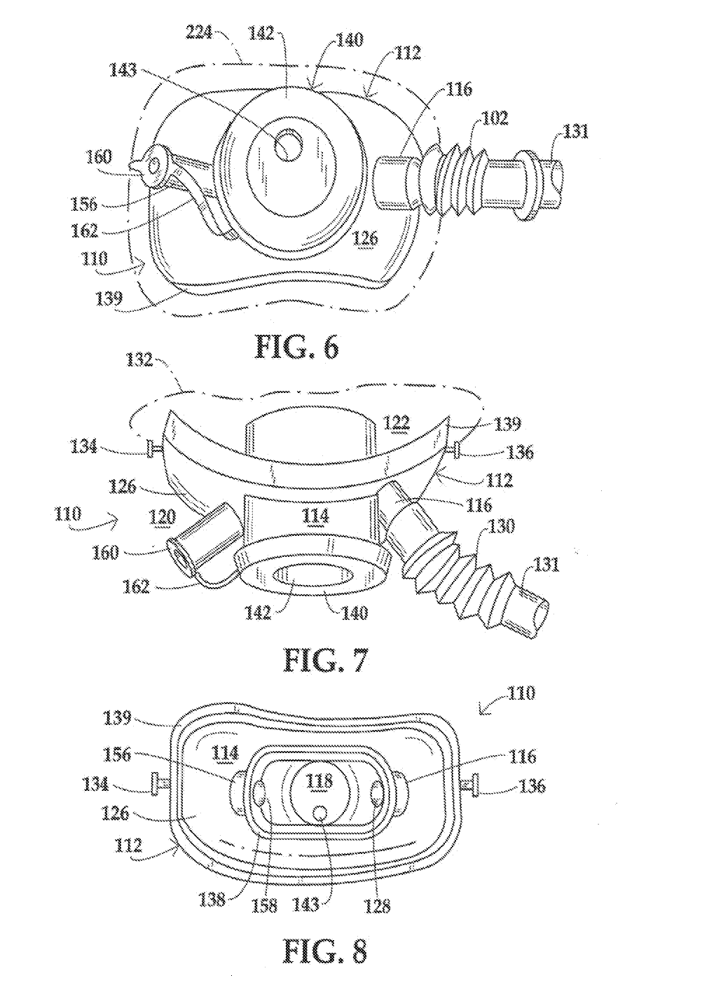

As illustrated FIGS. 6-8, a ventilating bite block 110 for facilitating upper gastro-intestinal (GI) endoscopic procedures comprises a body member 112 that includes a first tubular element 114 and a second tubular element 116. Tubular element 114 defines a first passageway, channel, or lumen 118 between a proximal side 120 and a distal side 122 of body member 112. Tubular element 114 is insertable into a patient's mouth between the teeth of the upper jaw and lower jaw. Passageway 118 serves as a deployment pathway for an endoscope insertion shaft 24 (FIG. 2).

Body member 112 further includes an arcuate flange 126 curved in two directions to form a shallow cup-shape connected to middle region of tubular element 114. Flange 126 is disposable outside and adjacent to the patient's mouth. Secondary tubular element 116 is also connected to flange 126 and defines a second passageway, channel or lumen 128 that serves to maintain an airway for ventilating the patient. Tubular airway element 116 is angled relative to and intersects tubular element 114 so as to communicate therewith. Tubular airway element 116 is connectable at a free or proximal end to a breathing apparatus (not shown) via a bellow-type connector 130 and an associated tube 131 for directing or guiding oxygen to the patient.

Bite block 110 may includes at least one elongate flexible tie member 132 (FIG. 7) such as a strap that is connectable to body member 112 at hooks, eyelets, or T-couplings 134 and 136. Strap 132 is extendible about the patient's head or neck for attaching body member 112 to the patient.

Flange 126 is formed with an endless sealing element 138 in the form of a rib extending about tubular port 114 and engageable with a patient's face about the port for forming a substantially airtight seal. Sealing rib 138 may be realized in any suitable manner including those discussed hereinabove with reference to FIGS. 1-5: closed cell foam or polymeric material, air-filled oval tube of a flexible and gas-impermeable polymeric composition, a gel-filled oval ring contained by a liquid-permeable bladder.

Flange 126 is additionally formed an inwardly turned peripheral lip 139 which may also engage the patient's face and serve as an additional seal. Lip 139 extends along the entire periphery or outer edge (not separately designated) of flange 126. Lip 139 may be an integrally formed part of flange 126 or may include, for instance, additional sealing material such as closed cell foam or polymeric material.

On an outer or proximal side of endoscope passageway 118, tubular element 114 is provided with a sealing element in the form of a cap 140 provided with a web or diaphragm 142 that extends across the one end of passageway 118. Web or diaphragm 142 is provided with an aperture 143 traversable by endoscope insertion shaft 24 (FIG. 2) and forming an airtight seal therewith. Web or diaphragm 142 permits easy scope introduction and prevents any significant leaks even under positive pressure ventilation.

Bite block 110 includes an additional tubular member 156 connected to flange 126 and defining a third passageway, channel or lumen 158 that serves as a path for introduction of a suction tube (not shown) or any other instrument that might prove useful in an operation. Tubular element 156 is also angled relative to tubular element 114 and intersects the same so as to communicate therewith. Tubular member 156 is provided with a removable closure or end cap 160 connected to cap 140 via a tie element 162. Tubular member 156 may be provided internally with a diaphragm (not illustrated) for maintaining an air seal. Alternatively, closure or end cap 160 may be formed with a self-sealing slit (not shown) traversable by a suction tube or other instrument.

Passageway 128 of tubular element 116 partially defines an oropharyngeal airway that, together with sealing element 138, cap 140, and closure 160, enables positive pressure ventilation and the maintenance of oxygen saturation in spite of hypoventilation.

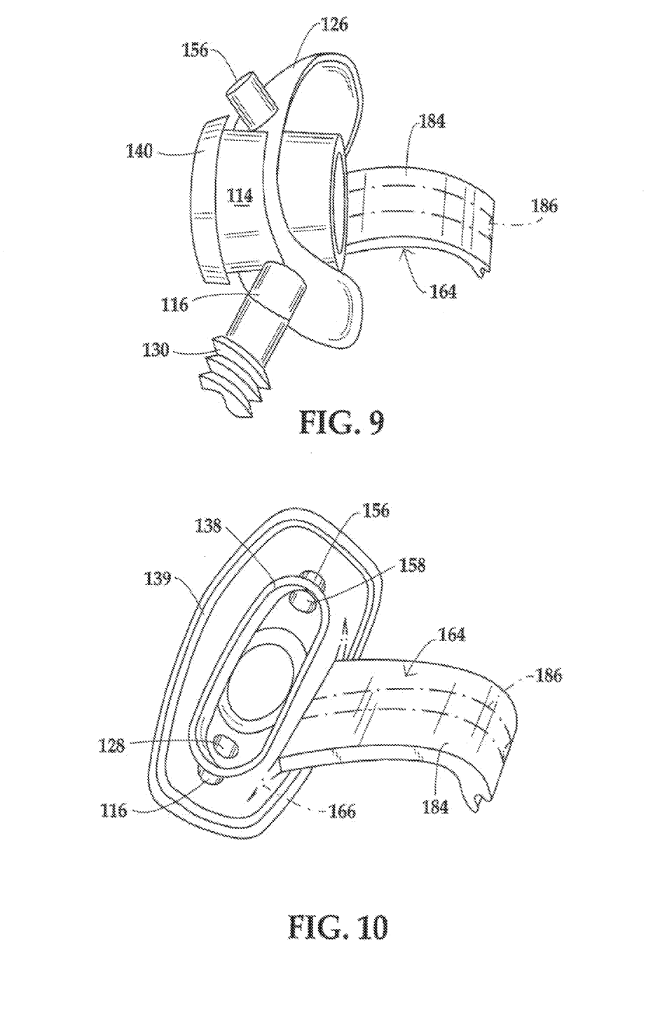

FIGS. 9 and 10 illustrate a modification of bite block 110 that includes a substantially rigid and slightly flexible arcuate extension 164 that serves to maintain an open airway or a patient prior to and after insertion of an endoscope shaft 24 (FIG. 2), an oropharyngeal or internal ventilation tube 44 (FIG. 2), and/or a ventilation tube member 58 (FIG. 5). Airway extension 164 may be fixed to flange 126. Alternatively, airway extension may be a separate piece attachable to body member 112 as needed, for instance, via a self-sealing slit 166 formed in flange 126 (FIGS. 8 and 10) or via hook-and-loop fasteners, nuts and bolts, hooks and eyelets, etc.

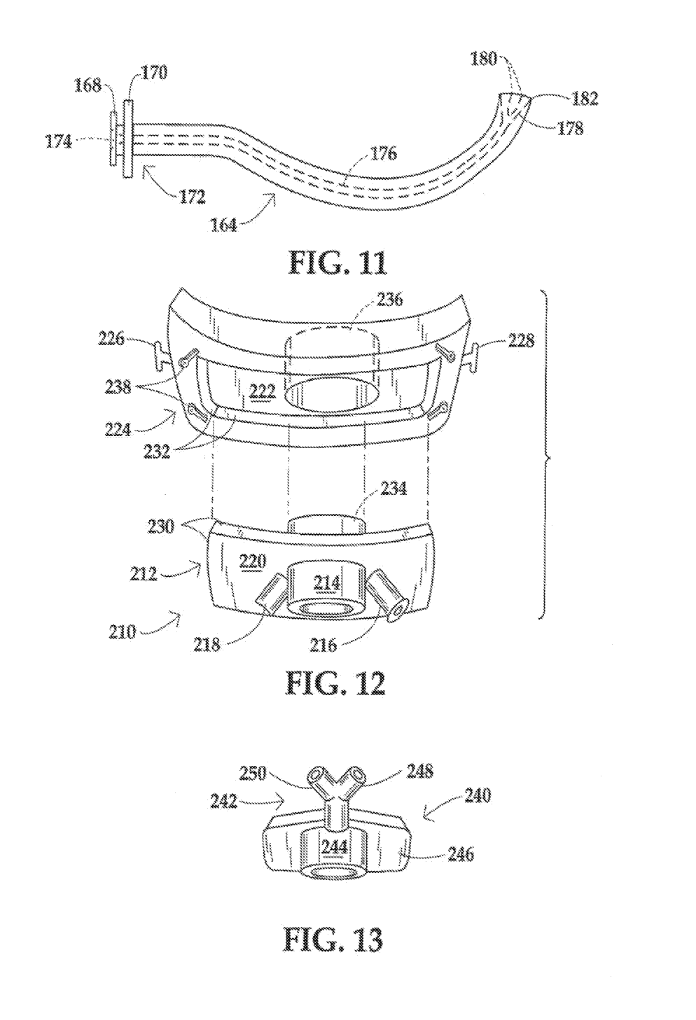

FIG. 11 depicts a specific form of airway extension 164 that includes a pair of flanges parallel flanges 168 and 170 at one end 172 for releasably securing the airway extension to body member 112 at slit 166, with the flanges being seated snugly on opposite sides of body member flange 126. Airway extension 164 is further provided at end 172 with a luer lock coupling 174. A thin-diameter channel or lumen 176 extends longitudinally through airway extension member 164 from luer lock 174 at end 172 to a manifold 178 of outlet channels 180 at a free end 182 of the airway extension, for enabling the spraying of an anesthetic composition into a patient. Manifold 178 and outlets 180 co-function as a nozzle. Airway extension 164 may be provided with luer lock 174, channel 176 and manifold 178 also where the airway extension is a fixed and permanent part of the bite block 110.

It is to be noted that airway extension 164 can also be used as a separate entity (on its own in various sizes for all age groups) in place of a traditional oropharyngeal airway. As illustrated in FIG. 11, such a stand-alone airway maintenance member may include luer lock coupling 174, channel or lumen 176 and one or more outlet channels 180 serving as a nozzle for enabling the spraying of an anesthetic composition or other liquid composition into a patient.

As indicated schematically in FIGS. 9 and 10, airway extension 164 may be formed in an upper surface 184 with a longitudinally extending groove 186 of semicircular cross-section for guiding endoscope or bronchoscope insertion shaft 24 during insertion thereof and for seating and retaining the endoscope shaft during a procedure.

FIG. 12 depicts a ventilating bite block assembly 210 for facilitating upper gastro-intestinal (GI) endoscopic procedures comprises a body member 212 that includes a first tubular port element 214 for endoscope access, a second tubular port element 216 for airway access, and a third tubular port member 218 for ancillary instruments such as a suction tube. Tubular port elements 214, 216, and 218 are connected to an arcuate flange 220 of body member 212 that is curved in two directions to form a shallow bowl-shape. Tubular port elements 214, 216, and 218 have structural features and functions discussed above with reference to tubular elements 114, 116, and 156. Body member 212 is removably seatable inside a recess 222 of an outer body or base member 224.

Outer body member 224 has hooks, eyelets, or T-couplings 226 and 228 to which a strap (not shown) may be connected for attaching the coupled body members 212 and 224 to a patient's head or neck.

Main body member 212 may be releasably fixed to outer body member 224 via a press-lock or friction fit. To that end, lateral surfaces 230 of main body member 212 snugly engage side walls 232 of recess 222. In addition, a distal side portion or extension 234 of tubular member 214 fits with close tolerance inside a tubular receptacle 236 of outer tubular member 224. Both main body member 212 and outer body member 224 are provided on distal sides with sealing elements (not shown) in the form of endless (oval, rectangular, circular) ribs, rings or gaskets exemplarily made of closed cell foam or polymeric material, gel or air filled tubes as described hereinabove. In the case of ribs, the sealing elements may be made of the same material as body members 212 and 224. Other means of removably coupling body members 212 and 224 to one another will occur to those skilled in the art. For instance, movable tabs 238 may be rotatably secured to outer body member 224 for locking the main body member 212 to the outer body member 224.

FIG. 6 schematically shows outer body member 224 as utilizable with bite block 110. FIG. 13 illustrates a ventilating bite block 240 like block 110 or 210, except that the airway port 116 and the suction port 156 of the latter are replaced with a single Y-port 242 extending in an upward direction from a tubular endoscope port 244 and a main body member or flange 246. The embodiment of FIG. 13 is intended for use with patients in a supine or face-down position on an operating table. Y-port 242 includes an airway leg or port channel 248 and an ancillary leg or port channel 250 for a suction instrument or other surgical tool. Y-port 242, as well as tubular ports 16, 116 and 156, may be removably connected to the respective body members 12, 112, 246. In that case, plugs or caps (not shown) are provided for sealing respective apertures in body members 12, 112, 246 vacated by the ports 16, 116 and 156 upon removal thereof.

Although the invention has been described in terms of particular embodiments and applications, one of ordinary skill in the art, in light of this teaching, can generate additional embodiments and modifications without departing from the spirit of or exceeding the scope of the claimed invention. For instance, where ventilation tube assembly 56 is used, tubular airway member 16 may be omitted. In that case, a simple aperture is provided in flange 26 to serve as the airway and cuff 56 inflates so as to fix ventilation tube member 58 to bite block 10 and to form an airtight coupling with the bite block.

It is contemplated that the various components of bite blocks 10, 110, 210, 240 disclosed herein may be combined with each other in different combinations to produce alternative embodiments of the invention. Different numbers of tubular access ports may be provided in different positions relative to one another. An air seal may be effectuated with various kinds of sealing elements. An airway extension 164 may be provided, which is either fixed to or removable from the bite block body member 12, 112 and which is either a solid piece or provided with a thin-diameter lumen 176.

Accordingly, it is to be understood that the drawings and descriptions herein are proffered by way of example to facilitate comprehension of the invention and should not be construed to limit the scope thereof.

1. A bite block comprising:
a body member including a first tubular element defining a first passageway between a proximal side and a distal side of said body member, said first tubular element being insertable into a patient's mouth between the teeth of the upper jaw and lower jaw, said body member further including a flange portion connected to said first tubular element, said flange portion being disposable outside and adjacent to the patient's mouth;
a second tubular element connected to said flange portion and defining a second passageway between said proximal side and said distal side of said body member, said second tubular element being connectable to a breathing apparatus for directing or guiding oxygen to the patient;
at least one tie or coupling member connectable at least indirectly to said body member for attaching said body member to the patient;
at least one sealing element on a distal surface of said flange portion for engaging the patient's face about the mouth in an at least substantially airtight fit; and
a diaphragm attached to said first tubular element and disposed across said first passageway for forming an airtight seal with an endoscope insertable through said first passageway.

2. The bite block defined in claim 1, further comprising a third tubular element connected at least indirectly to said flange portion and defining a third passageway between said proximal side and said distal side of said body member.

3. The bite block defined in claim 2 wherein said second tubular element and said third tubular element are parts of a Y-port.

4. The bite block defined in claim 2 wherein at least one of said second tubular element and said third tubular element is removably connected to said flange portion.

5. The bite block defined in claim 2 wherein said second tubular element and said third tubular element intersect and communicate with said first tubular element.

6. The bite block defined in claim 1, further comprising an arcuate airway extension projecting from said distal side of said body member.

7. The bite block defined in claim 6 wherein said airway extension is formed with a longitudinally extending lumen, a coupling port at one end for connecting said lumen to a source of fluid, and a nozzle at an opposite end.

8. The bite block defined in claim 6 wherein said body member is provided with a connection component for enabling a detachable coupling of said airway extension to said body member.

9. The bite block defined in claim 1, further comprising an outer base member, said body member being removably attachable to said base member, said tie or coupling member being connected to said base member.

10. The bite block defined in claim 1, further comprising a tubular member made of a partially flexible soft polymeric material and connectable to said second tubular element and extendible into the patient's pharynx to the entrance to the patient's trachea.

11. The bite block defined in claim 1 wherein said second tubular element extends at least partially in a proximal direction away from said flange.

12. The bite block defined in claim 1 wherein said second tubular element is rigidly connected to said flange portion.

13. An airway extension for a bite block, said airway extension having a longitudinally extending lumen, a coupling port at one end for connecting said lumen to a source of fluid, and a nozzle at an opposite end.

14. A medical method comprising:
providing a bite block including a body member defining a first passageway and a second passageway, said first passageway being provided with at least one first sealing element, said body member including a flange with at least one second sealing element;
attaching said body member to a patient so that said flange is disposed outside and adjacent to the patient's mouth and so that said at least one second sealing element engages the patient's face about the patient's mouth to form an airtight seal between said body member and the patient's face;
connecting said second passageway to a breathing apparatus;
thereafter operating said breathing apparatus to ventilate the patient; and
inserting an endoscope through said first passageway, said first sealing element forming an airtight seal with said endoscope.

15. The method defined in claim 14, further comprising attaching a tubular member to said body member so that said tubular member communicates with said second passageway; and inserting said tubular member into the patient's pharynx to the entrance to the patient's trachea.

16. The method defined in claim 14, further comprising operating said endoscope to view internal tissue structures of the patient while operating said breathing apparatus to ventilate the patient and maintaining said body member in airtight engagement with the patient's face and said endoscope.

17. A bite block including a body member defining a first passageway and a second passageway each extending between a proximal side and a distal side of said body member, said first passageway being provided with a first sealing element engageable with an outer surface of an endoscope insertion shaft for forming an airtight seal with the endoscope shaft, said second passageway being operatively connectable to a breathing apparatus for ventilating a patient, said body member including a flange with a second sealing element for engaging the face of a patient in an airtight fit about the mouth of the patient.

18. The bite block defined in claim 17 wherein said body member includes a tubular element connected to said flange portion and defining said second passageway.

19. The bite block defined in claim 17, further comprising an elongate tubular member made of a partially flexible soft polymeric material and connectable to said body member at said second passageway, said tubular member having sufficient length to extend from said body member at the patient's mouth into the patient's pharynx to the entrance to the patient's trachea.

20. The bite block defined in claim 19 wherein said elongate tubular member is provided with an inflatable cuff for coupling said elongate tubular member to said bite block with an airtight seal.

21. The bite block defined in claim 17, further comprising a third tubular element connected at least indirectly to said flange portion and defining a third passageway between said proximal side and said distal side of said body member.

22. The bite block defined in claim 21 wherein said second tubular element and said third tubular element are parts of a Y-port.

23. The bite block defined in claim 17, further comprising an arcuate airway extension projecting from said distal side of said body member.

24. The bite block defined in claim 17, further comprising an outer base member, said body member being removably attachable to said base member, said tie or coupling member being connected to said base member.
